(19) United States
(12) Patent Application Publication
(10) Pub. No.: US 2008/0020964 A1
(43) Pub. Date: Jan. 24, 2008

(54) Test System for the Discovery of Substances for the Promotion of Neuraxonal Growth

(76) Inventors: Michael Sendtner, Veitshochheim (DE); Wilfried Rossoll, Gerbrunn (DE); Sibylle Jablonka, Wurzburg (DE)

Correspondence Address: MAYER & WILLIAMS PC, 251 NORTH AVENUE WEST, 2ND FLOOR, WESTFIELD, NJ, 07090, US

(21) Appl. No.: 10/550,385

(22) PCT Filed: Mar. 16, 2004
(86) PCT No.: PCT/DE04/00577

§ 371 Date: Feb. 23, 2007

(30) Foreign Application Priority Data
Mar. 21, 2003 (DE) 103 12 846.8

(51) Int. Cl. A61K 31/00; A61P 25/00; C12Q 1/02; C12Q 1/68

(52) U.S. Cl. 514/1; 435/29; 435/6

(57) ABSTRACT

The invention relates to a method for identifying or testing active substances, which influence the growth and survival of nerve cells, wherein a cell is brought into contact with at least one substance and subsequently, within said cell, the mRNA of the β-actin or the β-actin protein alone or together with the SMN protein and/or a ribonucleic protein hnRNP, in particular the ribonucleic protein hnRNP-R and/or hnRNP-Q, are determined, and the determined amounts of said components are compared to the amounts of said components in a cell, which has not been brought into contact with the prospective active substance, to a test system for carrying-out said method and to uses of such test systems.

### FIELD OF THE INVENTION

The invention relates to a method and a test system for identifying or testing active substances, which influence, in particular promote the growth and/or survival of nerve cells, to active substances obtainable therewith, to uses of the test system and uses of the active substances.

### BACKGROUND OF THE INVENTION AND PRIOR ART

The functioning of nerve cells depends on the formation and conservation of operative neurites. Since a substantial number of neurodegenerative diseases (for instance Alzheimer's disease, diabetic neuropathy, multiple sclerosis and the damages after cerebral ischemia) are accompanied by degenerations of neurites, there is a significant need of substances, which promote the neuraxonal growth and inhibit the degeneration of neurites.

Ribonucleic proteins (RNP) are RNA-binding proteins, involved in the biogenesis, the transport and the function of mRNA and rRNA. To the RNP's belong the small nuclear ribonucleic proteins (snRNP) and the heterogeneous nuclear ribonucleic proteins (hnRNP). hnRNP's play an important role for various aspects of the RNA metabolism such as splicing, transport, polyadenylation, stabilization and translation (Review: Krecic and Swanson, 1999; literature list with complete bibliography see end of the description).

Some snRNP's are known for that they represent binding partners for the survival motor neuron (SMN) protein (Liu and Dreyfuss, 1996; Meister et al., 2000). SMN is found in a complex with some other proteins such as Gemin 2, Gemin 3, and Gemin 4, binds to U snRNA's (Fischer et al., 1997; Bühler et al., 1999; Selenko et al., 2001) and is involved in the biogenesis and functioning of snRNP's, the splicing process of the pre-mRNA and the processing and modification of rRNA (Pellizzoni et al., Curr Biol 11:1074-1088, 2001; Friesen et al., Mol Cell 7:1111-1117, 2001).

Newer findings show that special hnRNP's (hnRNP-R and hnRNP-Q's) interact with standard SMN proteins, whereas mutated SMN from patients with spinal muscular atrophy (SMA) does not bind to hnRNP's (Mourelatos et al., EMBO J 20:5443-5452, 2001). For these special hnRNP's, in turn, their involvement in the splicing process of the m-RNA was shown (Mourelatos et al., EMBO J 20:5443-5452, 2001).

Mutations or deletions of the gene for SMN lead to a reduction of functional SMN protein and thus to an occurrence of the spinal muscular atrophy (SMA), an autosomal recessive muscular disease, in the course of which a progressive muscular weakness and muscular atrophy by a progressive degeneration of the motor neurons is generated (Korinthenberg et al., 1997; Melki, 1997).

The causality is verified in so far as a reduced expression of SMN leads to the apoptosis of motoneurons of the anterior horn of the spinal cord and to SMA (Crawford and Pardo, 1996), and in mice to the degeneration of motoneurons (Jablonka et al., 2000).

However, in spite of this causality, the SMN protein seems not to be a survival factor for motoneurons, since an overexpression of the normal SMN protein does not protect motoneurons from cell death, for instance caused by withdrawal of trophic factors (Cisterni et al., Neurobiol Dis 8:240-251, 2001). Furthermore it is questionable, whether in the SMA the splicing process of the mRNA in motoneurons is affected (Jablonka et al., 2000; Tucker et al., 2001).

In spite of the progress in understanding the functioning of SMN in the pre-mRNA splicing, little only is known about the pathomechanism in the SMA. It is an open question, why reduced amounts of functional SMN protein in all tissues can lead to a specific death of motor neurons. A possible explanation could be that SMN fulfills additional motor neuron-specific tasks. This function could be based on the interaction with motor neuron-specific proteins. SMN is, besides in nucleic structures, also localized in the cytoplasm of motor neurons, including the dendrites and axons (Pagliardini et al., 2000).

The SMA belongs to the neurodegenerative diseases, which are characterized by the degeneration of nerve cells. Thereto belong Alzheimer's disease, diabetic neuropathy, multiple sclerosis and consequential diseases of the cerebral ischemia. A prophylaxis or therapy of these diseases is at present, if at all, to a limited degree only possible. New methods for identifying active substances and also active substances for the prophylaxis or therapy of these diseases are therefore urgently needed.

Newer findings (Rossoll et al., 2002) show that i) special hnRNP's, called hnRNP-R and hnRNP-Q, are expressed, although they can be found in the organism ubiquitously in cells, to the strongest degree during the embryogenesis in the spinal cord, ii) that hnRNP-R is substantially expressed in the cytoplasm of motoneurons, and that in particular in the axons thereof, less in axons of sensory neurons, and iii) that the overexpression of hnRNP-R or hnRNP-Q in nerve cells leads to a significantly increased growth of neurites.

### Technical Object.

It is therefore the object of the invention to provide a test system, by means of which active substances can be identified, which are suitable for the prophylaxis and/or treatment of neurodegenerative diseases and/or nerve damages by injuries, and to specify such active substances.

### Findings the Invention is Based on.

The basis of the invention is the surprising finding that the heterogeneous nuclear ribonucleic proteins R and Q (hnRNP-R and hnRNP-Q), which are known to substantially promote the growth of neurites, in common with the product of the SMN gene specifically bind to the mRNA of β-actin, and that this complex translocates into the axons and growth zones of motoneurons.

### Basics of the Invention and Preferred Embodiments.

Subject matter of the invention are thus substances, which lead to an increase of the formation of complexes of the following components in nerve cells, (1) hnRNP, in particular of hnRNP-R and hnRNP-Q and all their splicing isoforms, (2) SMN protein, and (3) β-actin mRNA, test systems for the discovery of such substances and also for the determination of the functional state of nerve cells, wherein β-actin alone (mRNA or protein) or in combination with at least one further component of this complex is detected, and the use of these substances for the prophylaxis and/or therapy of neurodegenerative diseases or nerve damages by injuries.

In a particular embodiment of this invention, these substances promote the complex formation of hnRNP-R and/or hnRNP-Q including all splicing isoforms (in the following only called hnRNP-R and -Q) with the SMN protein and with the mRNA for β-actin.

To these substances belong nucleic acid sequences, which code for ribonucleic proteins hnRNP-R and -Q, and which are introduced into the nerve cell by using methods known to the man skilled in the art, such as for instance by using viral vectors or non-viral vectors known to the man skilled in the art.

To these substances belong however also active substances, which act on the nerve cell such that the nerve cell forms to a stronger degree ribonucleic proteins, in particular hnRNP-R and/or hnRNP-Q proteins.

Subject matter of the invention are further methods for identifying active substances, which amplify the complex formation of ribonucleic proteins, in particular of hnRNP-R and/or hnRNP-Q in nerve cells, wherein a cell is brought into contact with the substance to be tested, and within the cell the amount of β-actin alone or with at least one further component of the complex is determined.

Subject matter of the invention is further the use of an active substance according to the invention for the diagnosis, the prophylaxis and/or therapy of a neurodegenerative disease or nerve damages by an injury or poisoning.

Subject matter of the invention is further the detection of β-actin alone or with at least one further component of the complex for the diagnosis and/or control of the course of a neurodegenerative disease. Such a detection occurs either molecular-biologically by means of nucleotide sequences, which specifically hybridize with the whole nucleotide sequence of β-actin or with parts thereof, for instance by using the in-situ hybridization or reverse transcriptase polymerase (RT-PCR) both known to the man skilled in the art, or the detection occurs by using antibodies or antibody fragments, which bind to actin or specifically β-actin, or by using other substances, which specifically bind actin, such as fluorescence-marked phalloidin or phallacidin.

With regard to methods for the detection or determination of one or several components of a test system according to the invention, explicit reference is made to the drawings.

The term ribonucleic proteins (RNP's) comprises in particular all snRNP's, including all splice variants or isoforms of human and non-human origin. Said term does not comprise non-functional mutations. Corresponding considerations apply to SMN proteins and β-actin. The term non-functional designates mutations, which do not show a complex formation RNP/SMN/β-actin mRNA in a test system otherwise according to the invention. The sequence of human hnRNP-R is known under the accession number AF000364.1. A newer version is known under the accession number NM_{—}005826. Sequences of human hnRNP-Q (3 splicing isoforms Q1, Q2 and Q3) are known under the accession numbers AY034483, AY034482 and AY034481, respectively. The sequence of mouse hnRNP-R is known under the accession number AF441128. The sequence of mouse hnRNP-Q is known under the accession number AF093821. The sequence of human SMN is known under the accession number AH006635. The sequence of human β-actin is known under the accession number NM_{—}001101 (mRNA). For the purpose of the invention, functional partial sequences and binding regions of the components can also be used. Thereto belong for instance the 3′ untranslated region of the β-actin mRNA and the zipcode region.

The galenic preparation of a pharmaceutical composition according to the invention may be performed in a usual way. As counter-ions for ionic compounds can for instance be used Na⁺, K⁺, Li⁺ or cyclohexyl ammonium. Suitable solid or liquid galenic preparations forms are for instance granulates, powders, dragees, tablets, (micro) capsules, suppositories, syrups, juices, suspensions, emulsions, drops or injectable solutions (IV, IP, IM), and preparations with protracted release of active substance, for the production of which usual means are used, such as carrier substances, explosives, binding, coating, swelling, sliding or lubricating agents, tasting agents, sweeteners and solution mediators. As auxiliary substances are named here magnesium carbonate, titanium dioxide, lactose, mannite and other sugars, talcum, milk protein, gelatin, starch, cellulose and derivatives, animal and vegetable oils such as codliver oil, sunflower oil, peanut oil or sesame oil, polyethylene glycols and solvents, such as sterile water and mono or multi-valent alcohols, for instance glycerin. A pharmaceutical composition according to the invention can be produced by that at least one substance used according to the invention is mixed in a defined dose with a pharmaceutically suitable and physiologically well tolerated carrier and possibly further suitable active, additional or auxiliary substances, and is prepared in the desired form of administration.

In the following, the invention is explained in more detail with reference to embodiments representing examples of execution only.

### EXAMPLE 1

### Measurement of the Axonal Growth of Motoneurons from a Mouse Model for Spinal Muscular Atrophy (SMA)

Primary motoneurons are isolated from the lumbal spinal cord of Smn−/−; SMN2 mice and Smn+/+; SMN2 controls. In this model for spinal muscular atrophy (SMA), human SMN2 is expressed (Monani et al., 2000) as a transgene in mice with deleted Smn (Schrank et al., 1997). The motoneurons are cultivated according to methods described already (Wiese et al., 1999). Spinal motoneurons were isolated at the embryonic day 14 because of their binding to culture dishes coated with anti-p75 neurotrophin-receptor antibodies and plated out in a density of 3,000 cells/cm² in 6-well culture dishes (Greiner Bio-One GmbH, Frickenhausen, Germany) on glass plates, which were coated with polyornithine and laminin (Wiese et al., 1999; Wiese et al., 2001). The cells were cultivated in neurobasal medium (Invitrogen, Carlsbad, Calif., USA) with horse serum, 500 μM glutamax and 50 μg/ml apotransferrin at 37° C. and 5% CO₂. The genotyping of the embryos took place according to a described method (Monani et al., 2000). CNTF and BDNF were added to obtain a final concentration of 10 ng/ml. Half the medium was replaced at the first day and every second day. For measuring the neurite length, the motoneurons were fixed, after having been cultivated for 5 days, with 4% paraformaldehyde in buffered common salt solution (PBS) and re-fixed with acetone. After blocking unspecific binding sites with 10% albumin from bovine serum (BSA), the cells were incubated over night at 4° C. with the following antibodies: rabbit antibodies against phospho-tau (Sigma, St. Louis, Mo., USA; 1 μg/ml) and a monoclonal mouse antibody against MAP-2 (Chemicon, Pittsburg, Pa., USA; 1:1,000). The cells were washed three times with Tris buffered common salt solution with Triton X-100 (TBS-T), incubated for 1 hour with Cy2 and Cy3-conjugated secondary antibodies (Dianova, Hamburg, Germany; 1:200) and after washing again with TBS-T, the cells were embedded in Mowiol (Sigma, St. Louis, Mo., USA).

The stained cells were scanned in at the confocal laser-scanning microscope (Leica, Solms, Germany). The staining with anti-phospho-tau identifies axonal extensions. The length of the extensions was measured with the Scion Image software (Scion Corp., Frederick, Md., USA).

The motoneurons obtained from Smn−/−; SMN2 mouse embryos showed a specific reduction of the length of the phospho-tau-positive axons (−27%) compared to Smn+/+; SMN2 controls (224.7±20.5 μm vs. 307.6±23.1 μm) . The result indicates a reduced axonal growth due to a reduced amount of Smn protein.

### EXAMPLE 2

### Measurement of the Neurite Growth in PC12 Cells Transiently Transfected with Smn or hnRNP-R and -Q

Rat pheochromocytoma cells (PC12) were cultivated in DMEM (Invitrogen, Carlsbad, Calif., USA)+10% horse serum+5% fetal calf serum+antibiotics. Before the transfection, the cells were plated out in a high density in 24-well culture dishes and transfected with 1 μg plasmid DNA (Lipofectamine 2000; Invitrogen, Carlsbad, Calif., USA). For this purpose, expression plasmids were used, into which wildtype forms marked with epitope tags (HA tag of FLAG tag) or mutated versions of Smn, hnRNP-R or hnRNP-Q have been cloned. On the day after the transfection, the cells were plated out in 35 mm culture dishes on glass plates coated with poly-DL-or nithine in a density of 1,000 cells/cm². As a differentiation medium, DMEM+2% horse serum and 1% fetal calf serum+50 ng/ml NGF+antibiotics were used. After three days, the cells were fixed and stained. The PC12 cells were fixed with 4% paraformaldehyde (PFA) in buffered common salt solution (PBS). After blocking unspecific binding sites with 15% goat serum+0.3% triton X-100, the cells were incubated over night at 4° C. with the following antibodies: rabbit antibodies against hnRNP-R (Rossoll et al., 2002; 1:1,000), anti-neurofilament H (Sigma, St. Louis, Mo., USA; 1:400), anti-HA (HA.11, Covance, Denver, Pa., USA; 1:250) and anti-FLAG M2 (Sigma, St. Louis, Mo., USA; 1:500). The cells were washed three times with Tris buffered common salt solution with Triton X-100 (TBS-T), incubated for 1 hour with Cy2 and Cy3-conjugated secondary antibodies (Dianova, Hamburg, Germany; 1:200). After washing again with TBS-T, the cells were embedded in Mowiol and evaluated at the fluorescence microscope or the confocal microscope as mentioned above.

Transiently transfected PC12 cells, which overexpressed the wildtype form of Smn, hnRNP-R or -Q, had an approx. 25-30% increase of neurite growth compared to the controls. Smn with a point mutation, which did not show any interaction with hnRNP-R (Rossoll et al., 2002) and which was also found in SMA patients (SmnY272C), had no stimulating effect. Mutated hnRNP-R with a deletion of the first two RNA binding domains (of amino acid 166-331) or the deletion of the Smn interaction domain (of amino acid 522-556) did not promote the neurite growth. A co-expression of the wildtype form of Smn with mutated hnRNP-R suppressed the growth-promoting effect. Conversely, the co-expression of the wildtype form of hnRNP-R with the mutated Smn also eliminated the growth-promoting effect.

In total these experiments show that the neurite growth-promoting effect of Smn and hnRNP-R only occurs for the wildtype forms, which can interact with each other.

### EXAMPLE 3

### Measurement of the Neurite Growth in Smn or hnRNP-R or -Q Overexpressing Stable PC12 Cell Lines

For preparing stable cell lines, PC12 cells were cultivated as described above, and transfected with expression vectors for the wildtype form or mutated Smn, hnRNP-R or hnRNP-Q. Thereafter, the cells were selected in a small cell density with the antibiotic G418 with regard to their neomycin resistance. Individual clones were tested by Western blot analysis for their stable expression of the constructs to be expressed. For measuring the neurite length, the cells were plated out as described above on coated glass plates, differentiated by the addition of differentiation medium and fixed, stained after seven days, and the neurite growth was quantified as described above.

PC12 cell lines, which overexpressed the wildtype form of hnRNP-R and -Q, showed approx three times longer neurites than the control cell lines being untransfected or transfected with the empty plasmid. Cell lines, which overexpressed hnRNP-R without RNA interaction domains or Smn interaction domain (see above), did not show any increased neurite growth. The effect was stronger that than in the transiently transfected cell lines, since the stable cell lines could be differentiated over a longer period of time (seven rather than three days).

### EXAMPLE 4

### Detection of Smn and hnRNP-R and of the β-actin Content in Axons and Growth Cones or Motoneurons

The cultivation of motoneurons and the fixation, staining and evaluation was performed as described above. As antibodies, monoclonal anti-Smn antibodies (BD Biosciences, Lexington, Ky., USA, 1:500), rabbit anti-hnRNP-R antibodies (Rossoll et al., 2002; 1:1,000), anti-actin (Roche, Bale, Switzerland, 1:200) and β-actin (Abcam, Cambridge, UK; 1:1,000) were used. Since in neurites mainly β-actin is localized, the results are similar for anti-actin and specifically anti-β-actin.

In motoneurons of control mice, the β-actin-specific staining was concentrated in the distal sections of the axons and growth cones. In motoneurons of Smn−/−; SMN2 mice, this staining was much less significant. The stainings with anti-actin and anti-β-actin antibodies showed a similar result. This result makes it probable that Smn is necessary for the localization of β-actin at the growth cones.

Immunocytochemistry with anti-hnRNP-R antibodies showed a staining along the neurites with an accumulation in the growth cones. In the Smn−/−; SMN2 motoneurons, the staining was weaker compared to the control motoneurons. This finding correlates with the observed exclusive localization of the hnRNP-R mutant without Smn interaction domain in the cell nucleus. The interaction of hnRNP-R with Smn seems to be required for the distal localization of hnRNP-R. hnRNP-R and Smn show a co-localization in the axons of the cultivated motoneurons.

### EXAMPLE 5

### Detection of Smn and hnRNP-R and of the β-actin Content in Neurites and Growth Cones of Cultivated Cell Lines

The cultivation of PC12 cell lines and the fixation, staining and evaluation was performed as described above. As antibodies, monoclonal anti-Smn antibodies (BD Biosciences, Lexington, Ky., USA, 1:500), rabbit anti-hnRNP-R antibodies (Rossoll et al., 2002; 1:1,000), anti-actin (Roche, Bale, Switzerland, 1:200) and β-actin (Abcam, Cambridge, UK; 1:1,000) were used. Since in neurites preferably β-actin is localized, the results are similar for anti-actin and specifically anti-β-actin.

The stainings showed in the PC12 cell lines overexpressing the wildtype form of hnRNP-R and in the control cell lines transfected with the empty plasmid a strong concentration of β-actin in the growth zones of the neurites. In cell lines, which overexpressed the deletion mutant without RNA binding domains, only a weak staining was measurable in the growth zones. This dominant-negative effect indicates that functional hnRNP-R is needed for the correct localization of β-actin. The wildtype forms of Smn and hnRNP-R showed a co-localization in the neurites of the cell lines.

### EXAMPLE 6

### Detection of the Binding of β-actin mRNA to hnRNP-R

The untranslated 3′ regions of β-actin from the stop codon to the poly-A sequence or the so-called zipcode region (Kislauskis et al., 1994) were amplified by RT-PCR and cloned into a plasmid with the binding site for the T7 RNA polymerase (pTZ19). Optionally, a poly-A tail of 30 nucleotides was added. (Used primers:

actinUTRfwd ATGAAAGCTTAGGCGGACTGTTACTGAGCTGC, actinUTRpolyAfullrev, ATGAGAATTC-T(30)-GTGTAAGGTAAGGTGTGCAC, actinUTRpolyAziprev, ATGA-GAATTC-T(30)-CTGCGCAAGTTAGGTTTTGTC, actinUTRfullrev, ATGAGGATCCGTGTAAGGTAAGGTGTGCAC).
The plasmids were linearized by digestion with EcoRI at the 3′ end, and were purified. 0.2 μg were used for the RNA synthesis by means of in vitro transcription with the radioactively marked nucleotide α32P-CTP (T7 Transcription Kit, MBI Fermentas, Vilnius, Lithuania). The marked transcribed RNA was purified by centrifugation by means of G-25 columns (Amersham, Piscataway, N.J., USA).

A cell line from human embryonic kidney cells (HEK 293) was transfected with expression constructs marked by HA epitope for the wildtype forms or mutated versions of Smn or hnRNP-R. After 48 hours, the cells were lysated in lysis buffer (25 mM Tris-HCl pH=8.0, 137 mM NaCl, 2 mM EGTA, 2 mM EDTA, 10% glycerol, 1% Triton X-100, 0.05% β-mercaptoethanol and protease inhibitors; Roche, Bale, Switzerland), and the HA-marked proteins were purifies by immunoprecipitation with anti-HA agarose (HA.11, Covance, Denver, Pa., USA). The immune complexes were washed five times with the lysis buffer and once with the RBB buffer (RBB: 10 mM Tris pH=7.5, 1.5 mM MgCl₂, 250 mM KCl, 2 mM DTT and 0.25% triton X-100) and incubated with the marked RNA for 30 minutes at room temperature in RBB buffer. The RNA-protein complexes were washed three times RBB buffer and RBB buffer+5 mg/ml heparin (Sigma, St. Louis, Mo., USA) . The pellets were resuspended in 20 μl RBB buffer, and applied on nitrocellulose filters (Protran, Schleicher & Schuell BioScience GmbH, Dasse/Relliehausen, Germany). These filters were exposed with phosphoimager plates (BAS-2500, Photo Film Europe GmbH, Duesseldorf, Germany), and the bound radioactivity was measured as photo-stimulating luminescence (PSL) by means of the AIDA software package (Raytest, Straubenhardt, Germany).

The results show that the wildtype form, however not the mutated versions (see above) of hnRNP-R bind to the 3′ untranslated region of β-actin mRNA. This binding took place independently from the poly-A tail. The zipcode region alone shows specific binding, too.

These results not only demonstrated a binding of hnRNP-R to β-actin mRNA, but also suggested that the interaction with Smn is necessary for this binding, since the mutated version without the Smn interaction domain did not show any β-actin mRNA binding.

Altogether, the results indicate that a complex of Smn and hnRNP-R or -Q binds to β-actin mRNA and translocates into axons of motoneurons or also other neurites. Disturbances of these complexes will lead to reduced β-actin concentrations at the growth zones of the neurites and thus to a reduced axonal growth.

### LITERATURE

Buhler, D. et al., 1999, Hum. Mol. Genet., 8, 2351-2357.
Cisterni, C. et al., 2001, Neurobiol. Dis., 8, 240-251.
Crawford, T. O. et al., 1996, Neurobiol. Dis., 3, 97-110.
Fischer, U. et al., 1997, Cell, 90, 1023-1029.
Friesen, W. J. et al., 2001, Mol. Cell, 7, 1111-1117.
Jablonka, S. et al., 2000, Hum. Mol. Genet., 9, 341-346.
Kislauskis, E. H. et al., 1994, J. Cell Biol., 127, 441-451.
Korinthenberg, R. et al., 1997, Ann. Neurol., 42, 364-368.
Krecic, A. M. et al., 1999, Curr. Opin. Cell Biol., 11, 363-371.
Lefebvre, S. et al., 1995, Cell, 80, 155-165.
Lefebvre, S. et al., 1997, Nat. Genet., 16, 265-269.
Liu, Q. et al., 1996, EMBO J., 15, 3555-3565.
Meister, G. et al., 2000, Hum. Mol. Genet., 9, 1977-1986.
Monani, U. R. et al., 2000, Hum. Mol. Genet., 9, 333-339.
Mourelatos, Z. et al., 2001, EMBO J., 20, 5443-5452.
Pellizzoni, L. et al., 2001, Curr. Biol., 11, 1079-1088.
Rossoll, W. et al., 2002, Hum. Mol. Genet., 11, 93-105.
Schrank, B. et al., 1997, Proc. Natl. Acad. Sci. U.S.A, 94, 9920-9925.
Selenko, P. et al., 2001, Nat. Struct. Biol., 8, 27-31.
Tucker, K. E. et al., 2001, J. Cell Biol., 154, 293-307.
Wiese, S. et al., 1999, Eur. J. Neurosci., 11, 1668-1676.
Wiese, S. et al., 2001, Nat. Neurosci., 4, 137-142.

1. A method for the discovery or testing of active substances, which influence the growth and survival of nerve cells, wherein a cell is brought into contact with at least one active substance, wherein subsequently, within said cell, the mRNA of the β-actin or the β-actin protein alone or together with the SMN protein and/or a ribonucleic protein hnRNP, in particular the ribonucleic protein hnRNP-R and/or hnRNP-Q, are determined, for instance in growth zones of neurites, wherein the determined amounts of said components are compared to the amounts of said components in a cell, which has not been brought into contact with the prospective active substance, and in case of an increased amount, the active substance is selected.

2. A method according to claim 1, wherein one, several, or all components are detected and optionally quantitatively determined with antibodies or antibody fragments.

3. A method according to claim 1, wherein β-actin is detected and optionally quantitatively determined with actin-binding substances.

4. A method according to claim 1, wherein one, several, or all components are detected and optionally quantitatively determined by measurement of their mRNA.

5. A method according to claim 1, wherein the cell is a nerve cell.

6. A method according to claim 1, wherein the cell has a mesodermal or endodermal origin.

7. A test system for carrying-out a method according to claim 1, comprising a cell, cells of a cell line or a tissue sample containing cells, means for cultivating the cells, and means for qualitatively or quantitatively determining one, several, or all components.

8. The use of a test system according to claim 7 for the discovery of active substances, which promote the growth and survival of nerve cells and/or which promote the growth of neurites and axons.

9. The use of a test system according to claim 7 for testing the growth and survival of nerve cells, in particular for the diagnosis and/or control of the course of neurodegenerative diseases and/or nerve damages by injuries or poisoning.

10. An active substance obtainable with a method according to claim 1, wherein said active substance increases in a cell the amount of complexes containing the components ribonucleic proteins, in particular hnRNP-R and/or hnRNP-Q, SMN and β-actin mRNA.

11. The use of an active substance according to claim 10 for preparing a pharmaceutical composition for promoting the growth and the survival of nerve cells, in particular for the prophylaxis or therapy of neurodegenerative diseases and/or nerve damages by injuries or poisoning.
